(19) United States
(12) Patent Application Publication
(10) Pub. No.: US 2014/0316281 A1
(43) Pub. Date: Oct. 23, 2014

(54) NOISE SUBTRACTION FOR INTRA-BODY FIBER OPTIC SENSOR

(71) Applicant: Vascular Imaging Corporation, Rancho Cordova, CA (US)

(72) Inventors: Michael J. Eberle, Fair Oaks, CA (US); Howard Neil Rourke, Sacramento, CA (US); Diana Margaret Tasker, Fair Oaks, CA (US)

(73) Assignee: Vascular Imaging Corporation, Rancho Cordova, CA (US)

(21) Appl. No.: 14/104,069

(22) Filed: Dec. 12, 2013

Related U.S. Application Data
(60) Provisional application No. 61/737,299, filed on Dec. 14, 2012.

(51) Int. Cl. A61B 5/00

(52) U.S. Cl. CPC A61B 5/0084; A61B 5/742; A61B 5/7203
USPC 600/478

(57) ABSTRACT

An optical source can generally provide optical energy having phase noise. Such phase noise, when demodulated using an intravascularly-deliverable optical fiber transducer, can be indistinguishable from a signal of interest. Apparatus or techniques can include using one or more of a reference optical cavity or a delay line, such as to obtain information indicative of the phase noise of the optical source. Such information can then be reduced or suppressed from other information obtained from the intravascularly-deliverable optical fiber transducer, such as to improve a signal-to-noise (SNR) ratio of a sensing system including the intravascularly-deliverable optical fiber transducer.

This application claims the benefit of priority of U.S. Provisional Application No. 61/737,299, titled “NOISE SUBTRACTION FOR INTRA-BODY FIBER OPTIC SENSOR,” by Rourke et al., and filed on Dec. 14, 2012, the entire content of which being incorporated herein by reference.

### BACKGROUND

Interventional medical procedures are increasingly relied upon to treat patients suffering from coronary artery disease or cardiac ischemia. In particular, such patients may be treated using vascular bypass operations such as Coronary Artery Bypass Graft (CABG), or less invasive techniques. Less invasive techniques may include one or more of angioplasty or stenting, which can be referred to generally as Percutaneous Coronary Intervention (PCI). Other less invasive techniques can include atherectomy, or brachytherapy, for example. Such less invasive techniques often include introduction of one or more catheters or guidewires into the vasculature. Similar approaches can be used for treating various symptoms or diseases involving other physiologic locations (e.g., a biliary location, a carotid artery location, or one or more other locations). Various imaging techniques can provide useful feedback to a caregiver before, during, or after an intravascular or intraluminal procedure. Such imaging techniques can include X-ray (e.g., fluoroscopy) or ultrasound-based techniques.

Vardi & Spivak, U.S. Pat. No. 6,659,957, U.S. Pat. No. 7,527,594, and U.S. Pat. Pub. No. US-2008-0119739-A1, each of which is hereby incorporated by reference herein in its entirety, describe, among other things, an elongated imaging apparatus, for internal patient imaging, the apparatus including an electrical-to-acoustic transmit transducer and an acoustic-to-optical receive transducer.

Bates & Vardi, U.S. Pat. No. 7,245,789, U.S. Pat. No. 7,447,388, U.S. Pat. No. 7,660,492, U.S. Pat. No. 8,059,923, and U.S. Pat. Pub. No. US-2012-0108943-A1, each of which is hereby incorporated by reference herein in its entirety, describe, among other things, an elongated imaging apparatus, for internal patient imaging, the apparatus including an optical-to-acoustic transmit transducer and an acoustic-to-optical receive transducer.

### Overview

An optical fiber transducer can be configured to couple optical energy from an optical source to a sensing region along the transducer. The sensing region along the transducer can include an interferometer structure. The optical fiber transducer can be sized and shaped for intravascular use or for use in one or more other body lumens or locations.

Optical energy coupled to the interferometer structure can be modulated or otherwise adjusted in relation to one or more physiologic parameters (e.g., modulated by a pressure), or in relation to received acoustic energy (e.g., ultrasonic energy) or mechanical vibration. The optical fiber transducer can be included as a portion of a guidewire assembly. Ultrasound-based imaging or sensing, such as using the optical fiber transducer, can provide a variety of advantages as compared to other imaging or sensing techniques, or can complement other techniques. Unlike X-ray imaging techniques, ultrasound is an acoustic technique and is therefore non-ionizing.

A pressure or vibration coupled to a sensing region of the optical fiber transducer can induce a change in the refractive index of the optical fiber transducer, or can physically modulate an optical path length of optical energy within the interferometer, causing a detectable change in a reflected intensity of the optical energy. The modulated reflected optical energy from the interferometer can be coupled back to a detector, and processed, such as to determine one or more physiologic parameters or to construct an image.

A tunable optical source (e.g., a laser) can provide the optical energy to the interferometer structure. However, the optical energy generally includes small variations in wavelength (e.g., due to a laser's non-zero linewidth or phase noise). Such variations can be unwanted, and can produce demodulated noise in a signal detected using an interferometer structure such as the optical fiber transducer (e.g., the phase noise can be converted to amplitude noise). The demodulated noise can be indistinguishable from detected information generated by coupled acoustic or mechanical vibrational energy of interest, which can degrade a sensing system signal-to-noise (SNR) ratio. In an ultrasound imaging application, in particular, such noise can significantly degrade image quality. For example, in an ultrasound imaging application, even highly echogenic structures may only return about 1% of incident acoustic energy as reflected acoustic energy. Thus, any noise contribution from linewidth or phase noise of the optical source can be significant in comparison to a received signal of interest representative of the reflected acoustic energy.

The present inventors have recognized, among other things, that information indicative of the phase noise of the optical source can be obtained, such as corresponding to an instance of optical energy used to excite an interferometer structure. Information indicative of the phase noise can be used to process (e.g., enhance) the information of interest.

For example, the present inventors have also recognized that the intravascularly-deliverable optical fiber transducer can be configured to reflect a portion of optical energy modulated in response to a vibration, pressure, or strain, and such a transducer will generally also reflect a portion of the optical energy indicative of a phase noise of the first optical source. A reference optical cavity can be configured to reflect a portion of optical energy indicative of the phase noise of the first optical source less modulated or un-modulated by the vibration, pressure, or strain. Information obtained from the reference optical cavity (e.g., information indicative of phase noise) can then be subtracted or otherwise reduced or suppressed from other information obtained from the intravascularly-deliverable optical fiber.

In another example, a delay line (e.g., a line including a long optical fiber segment), including a length defining a specified optical propagation delay, can be coupled to an intravascularly-deliverable optical fiber transducer. During respective durations, optical energy provided by an optical source can be coupled to the optical fiber transducer respectively bypassing the delay line during a first duration, and traversing the delay line, during a second duration. In one respective duration, the optical energy reflected from the optical fiber transducer can include optical energy modulated in response to a vibration, pressure, or strain along with optical energy indicative of a phase noise of a first optical source. During another respective duration, the optical energy reflected from the optical fiber transducer can include optical energy indicative of the same instance of phase noise of the optical source but less modulated or un-modulated by the vibration, pressure, or strain. Similar to the previous example, information indicative of the phase noise can be subtracted or otherwise reduced or suppressed from other information obtained from the intravascularly-deliverable optical fiber.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1A illustrates generally an example that can include a portion of an optical fiber transducer, such as sized and shaped for delivery to an intravascular location.

FIG. 1B illustrates generally an example that can include a portion of an optical fiber transducer, such as sized and shaped for delivery to an intravascular location.

FIG. 2 illustrates generally an example that can include a portion of an optical fiber transducer, such as including a blazed grating structure, such as configured for use with an ultrasound technique.

FIG. 3 illustrates generally an illustrative example of optical energy, such as can be reflected from an interferometer structure.

FIG. 4 illustrates generally an illustrative example of a normalized signal of interest as compared with a noise signal.

FIG. 5 illustrates generally an example of a system that can include an intravascularly-deliverable optical fiber transducer and a reference optical cavity.

FIG. 6 illustrates generally an example of a system that can include intravascularly-deliverable optical fiber transducer and an optical delay line.

FIG. 7 illustrates generally an example of a system that can include an intravascularly-deliverable optical fiber transducer and a reference optical cavity.

FIG. 8 illustrates generally an illustrative example of at least a portion of a system that can include respective detectors coupled to an analog operational circuit.

FIG. 9 illustrates generally an example of a system that can include intravascularly-deliverable optical fiber transducer and an optical delay line.

FIGS. 10A through 10C illustrate generally illustrative examples of a noise signature that can be obtained from a sensing channel, a noise signature that can be obtained from a reference channel, and a relative indication of information that can be obtained such as using a difference between the signatures obtained from the sensing and reference channels.

FIGS. 11A through 11C illustrate generally illustrative examples of a signal of interest plus a noise signature that can be obtained from a sensing channel, a noise signature that can be obtained from a reference channel, and a relative indication of information that can be obtained such as using a difference between the signatures obtained from the sensing and reference channels.

FIG. 12A illustrates generally an illustrative example of noise reduction performance in decibels as a function of a timing alignment error between phase noise information in a signal of interest as compared to a phase noise obtained using a reference cavity or delay line.

FIG. 12B illustrates generally an illustrative example of noise reduction performance as a function of a amplitude normalization error between phase noise information in a signal of interest as compared to a phase noise obtained using a reference cavity or delay line.

FIG. 13 illustrates generally a technique, such as a method, that can include processing information about a vibration, pressure, or strain modulating optical energy reflected from an intravascularly-deliverable optical fiber transducer using information about a phase noise of an optical source obtained from a reference optical cavity.

FIG. 14 illustrates generally a technique, such as a method, that can include processing information about a vibration, pressure, or strain modulating optical energy reflected from an intravascularly-deliverable optical fiber transducer using information about a phase noise of an optical source obtained as least in part using an optical delay line.

### DETAILED DESCRIPTION

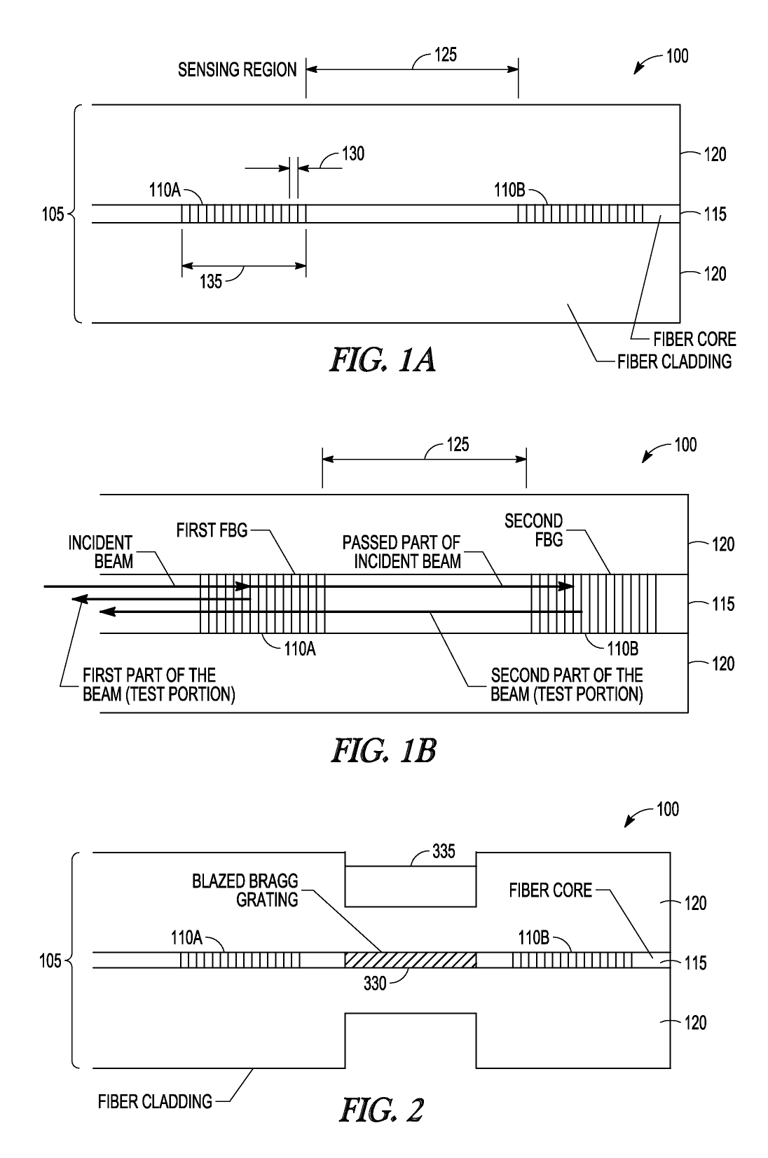

FIG. 1A illustrates generally an example that can include a portion of an optical fiber transducer 100, such as sized and shaped for delivery to an intravascular location. The optical fiber transducer 100 can include an optical fiber assembly 105, such as including an optical fiber core 115 and a cladding 120. The optical fiber transducer 100 can include an interferometer structure, such as comprising one or more Fiber Bragg Gratings (FBGs). An FBG can be configured to reflect a specified proportion of incident optical energy for a specified range of wavelengths, similarly to a mirror.

A first FBG 110A can be located along the optical fiber core. The first FBG 110A can include a specified or periodic variation in the index of refraction along a long axis of the optical fiber core 115. For example, the optical fiber core 115 can have a first index of refraction, and the first FBG 110A can include portions having a second index of refraction “written” or otherwise impressed in the optical fiber core 115 in a periodic configuration, such as having a spacing between the portions having the second index that can be referred to as the period 130 of the first FBG 110A. The first and second indices of refraction, and the period 130 of the first FBG 110A, can be used to control a range of wavelengths for which the first FBG 110A is reflective. The first and second indices of refraction and an axial length 135 of the first FBG 110A can be used to control a proportion of incident optical energy that is reflected versus transmitted through the first FBG 110A. A second FBG 110B can be located along the fiber core 115, such as separated from the first FBG 110A by a sensing region 125. A combination of the first and second FBGs 110A and 110B can establish an interferometer structure (e.g., a Fabry-Perot cavity).

As shown in FIG. 1B, incident optical energy (e.g., an incident beam) can be coupled to the interferometer structure established by the first and second FBGs 110A and 110B. Such optical energy can be generated by a first optical source, such as a laser. Along the length of the optic fiber transducer, the first FBG 110A can be located more proximally to the first optical source than the second FBG 110B.

A portion of the incident optical energy can be reflected by the first FBG 110A (e.g., a “first part of the beam”) and from the second FBG 110B (e.g., a “second part of the beam”). A phase relationship between the reflected portions of the optical energy can be adjusted such as by any change affecting the optical path length between the first and second FBGs 110A and 110B, such as an optical path including the sensing region 125. Such a change in optical path length can occur when one or more vibration, pressure, or strain is imparted on the optical fiber core 115 such as via the cladding 120, causing a change in the index of refraction of the optical fiber core 115 in the sensing region 125 or a physical lengthening or shortening the optical path in the sensing region 125. Such variation in the optical path length in the sensing region 125 can modulate or adjust an intensity of optical energy reflected from the interferometer structure. In this manner, the optical fiber transducer 100 can be configured to provide an acousto-optical transducer.

FIG. 2 illustrates generally an example that can include a portion of an optical fiber transducer 100, such as can include a “blazed” grating structure 330, such as configured for use with an ultrasound technique. As in the examples of FIGS. 1A and 1B, the optical fiber transducer 100 can be intravascularly deliverable, such as including an optical fiber assembly 105 including an optical fiber core 115, a cladding 120, and one or more FBGs such as first FBG 110A or a second FBG 110B. The first or second FBGs 110A or 110B can generally include a specified or periodically-varying index of refraction, with the index of refraction homogeneous in the radial direction, and varying in the axial direction along a longitudinal axis of the optical fiber core 115. In the example of FIG. 2, a “blazed” FBG can be included, such as having an index of refraction impressed obliquely at a non-perpendicular angle to the longitudinal axis of the optical fiber core 115. Such a blazed grating structure 330 can be used to outcouple optical energy from the optical fiber core 115. In an example, optical energy can be provided to the blazed grating structure 330 such as coupleable to an optically-absorptive photoacoustic material 335. Such an optically-absorptive photoacoustic material 335 can be specified to expand in the presence of intense optical energy, and can abruptly contract as the optical energy outcoupled from the optical fiber core 115 is reduced or suppressed, such as to provide an ultrasonic wave or other mechanical wave or vibration without requiring an electrically-driven transducer in the optical fiber transducer 100. Reflected acoustic or mechanical energy can then be sensed using an interferometer configuration including the first and second FBGs 110A and 110B, as discussed above in the examples of FIGS. 1A and 1B.

FIG. 3 illustrates generally an illustrative example 300 of optical energy, such as can be reflected from an interferometer structure, such as shown and described in one or more of the examples of FIGS. 1A, 1B, FIG. 2, or elsewhere herein. In the example of FIG. 3, an interferometer structure (e.g., a Fabry-Perot cavity) can provide one or more transmission features, such as a null 304 established by an optical path length between respective FBG structures. As an optical path length between the respective FBG structures varies (e.g., due to an impressed vibration, pressure, or strain), a location of the null 304 can shift in wavelength.

A tunable optical source can provide an output wavelength 302A locked to a specified region 306 of the null 304, such as to provide a desired or specified level of sensitivity to shifts in the null 304 location. Such wavelength shifts of the null 304 can be converted into variations in the amplitude or intensity of reflected optical energy (e.g., amplitude modulating the incident optical energy to provide the reflected optical energy) because the output wavelength 302A of the tunable optical source can remain roughly constant during such shifts.

The tunable optical source (e.g., a laser that can be coupled to an optical fiber transducer including the interferometer) can be controlled in part using a feedback loop configured to adjust an output wavelength 302A of the tunable optical source to keep the wavelength 302A aligned with the specified region 306 of the null 304 as the null 304 shifts for reasons not related to physiologic information of interest. For example, in an acoustic (e.g., ultrasound) or mechanical vibration sensing application, a loop bandwidth of one or more such feedback loops can be established below a frequency range corresponding to received acoustic or mechanical vibrational energy, so that the optical energy source tracks shifts in the null 304 location that are unrelated to the acoustic or mechanical vibrational energy. For frequencies above the specified loop bandwidth, the output wavelength 302A of the tunable optical source can remain roughly constant, and the null 304 location can shift around the output wavelength 302A in response to the coupled acoustic or mechanical vibrational energy and thereby can modulate the optical energy reflected by the interferometer. In an illustrative example of an ultrasound imaging application, the loop bandwidth can be about 1 kiloHertz (kHz).

In an illustrative example, the output wavelength 302A can be locked to a specified region 306 about halfway along a transition between relative minimum intensity of the optical energy reflected from the transducer and a relative maximum intensity of the optical energy reflected from the transducer. In another illustrative example, the output wavelength 302A can be locked to a region of about 30 to about 40 percent of the difference between the relative maximum intensity and the relative minimum intensity of the reflected optical energy.

The present inventors have recognized, among other things, that the tunable optical source (e.g., the laser) can provide an output that includes small variations in wavelength, such as randomly shifting the output wavelength 302A to other locations such as a different location 302B along the null 304. Such wavelength variation (e.g., due to a laser linewidth or phase noise) may be unwanted, and may be demodulated against the slope of the null 304, being transformed from phase noise into amplitude noise in a detected response reflected from the interferometer. The amplitude noise may be indistinguishable from shifts in the null 304 location caused by coupled acoustic or mechanical vibrational energy of interest. Such noise may be referred to generally as a “phase noise signature” or just “phase noise” elsewhere herein, even though such noise may manifest itself as amplitude variation in detected information.

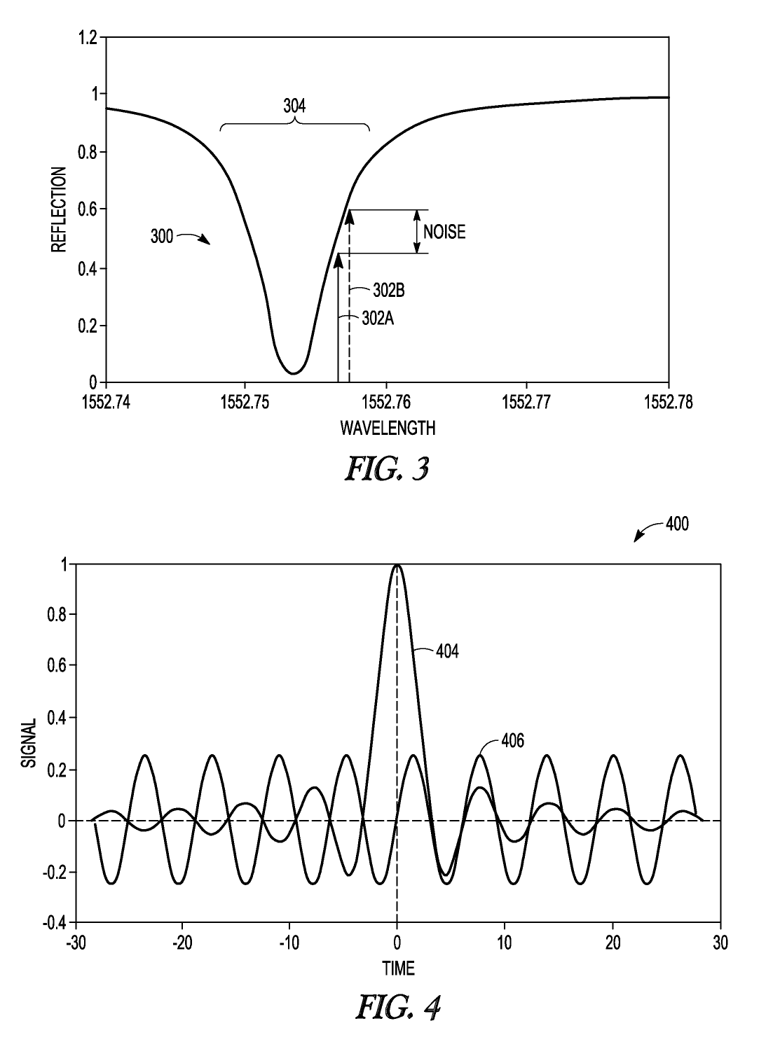

FIG. 4 illustrates generally an illustrative example of a normalized signal of interest 404 as compared with a noise signal 406. The noise signal 406 can include a time-varying amplitude that corresponds to a time-varying deviation in an output wavelength (or correspondingly, an output frequency) of a tunable optical source. For example, such as discussed in the example of FIG. 3, a laser output wavelength can vary, and an interferometer structure can demodulate such wavelength variation.

The noise signal 406 can be a significant proportion, or even larger than, the amplitude of the signal of interest 404. The signal of interest 404 can include information indicative of acoustic or mechanical vibrational energy coupled to an interferometer, and may include frequencies that overlap with a spectrum of the noise signal 406. Thus, zonal filtering (e.g., low-, band-, or high-pass filtering) of received information, in the frequency domain, generally does not suppress or eliminate the noise signal 406 while preserving the signal of interest 404. Accordingly, the present inventors have recognized, among other things, that a time-domain noise reduction or elimination technique can be used, such as subtracting a representation of the noise signal 406 from another signal that includes both information of interest 404 and the noise signal.

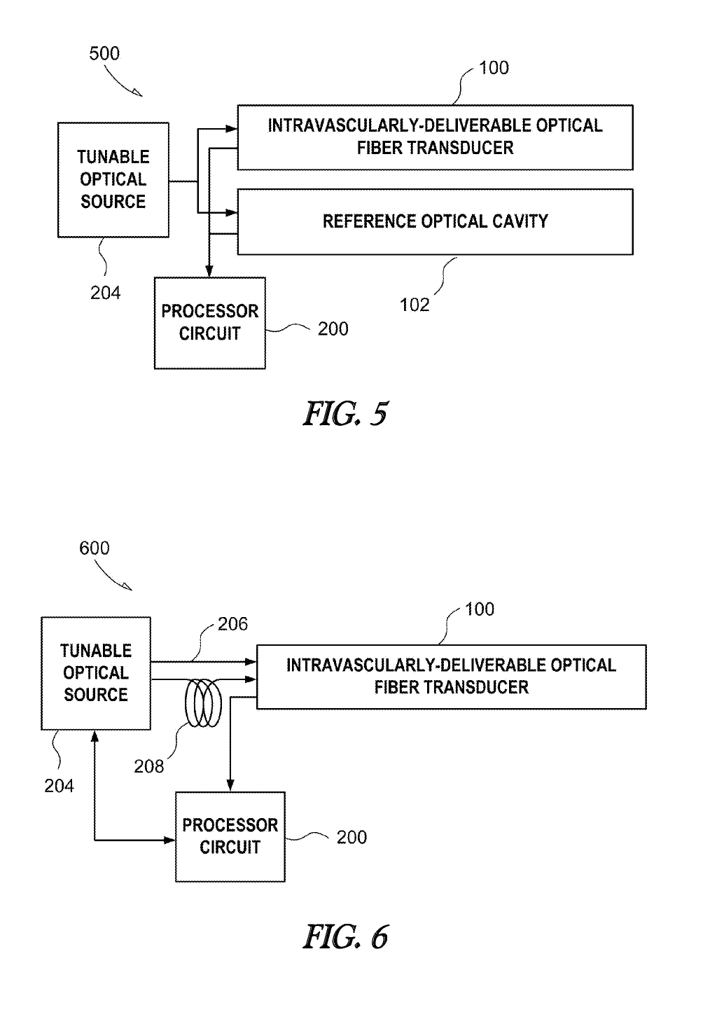

FIG. 5 illustrates generally an example of a system 500 that can include an intravascularly-deliverable optical fiber transducer 100, such as shown and described in the examples of FIGS. 1A, 1B, FIG. 2, or elsewhere herein. The system can include a tunable optical source 204, such as coupled to the intravascularly-deliverable optical fiber transducer 100. As discussed in the examples above and below, the tunable optical source 204 can be configured to provide optical energy at a specified output wavelength, and the intravascularly-deliverable optical fiber transducer 100 can modulate or otherwise adjust an intensity of the optical energy reflected from the intravascularly-deliverable optical fiber transducer 100. The optical energy from the tunable optical source 204 generally includes phase noise, such as corresponding to a finite non-zero linewidth of an optical output in the frequency domain.

Optical energy generated by the tunable optical source 204 can be coupled to a reference optical cavity 102, such as contemporaneously coupled to the intravascularly-deliverable optical fiber transducer 100 and the reference optical cavity 102. The reference optical cavity 102 can include an optical fiber transducer assembly (e.g., similar or identical to the intravascularly-deliverable optical fiber transducer 100) or other another assembly such as an ultra-high-finesse optical cavity. For example, a temperature of the reference optical cavity 102, or a region surrounding the reference optical cavity 102, can be controlled or stabilized. The reference optical cavity 102 can be tunable, such as using a temperature adjustment, such as to align one or more of a null or other transmission or reflection feature of the reference optical cavity 102 with a corresponding transmission or reflection feature of the intravascularly-deliverable optical fiber transducer 100.

Optical energy reflected from the intravascularly-deliverable optical fiber transducer 100 can include both of optical energy modulated in response to a vibration, pressure, or strain, and optical energy indicative of a phase noise of the first optical source (e.g., demodulated by the optical fiber transducer). Optical energy reflected from the reference optical cavity 102 can include optical energy indicative of the phase noise of the first optical source, but less modulated or un-modulated by the vibration, pressure, or strain. In this manner, the optical energy reflected from the reference optical cavity 102 can provide a phase noise “baseline” or profile that can be used to enhance a signal-to-noise ratio of information obtained from the intravascularly-deliverable optical fiber transducer 100, using one or more techniques described elsewhere herein.

For example, information indicative of reflected optical energy can be provided to a processor circuit 200, such as can be used to process information indicative of a vibration, pressure, or strain modulating the optical energy reflected from the intravascularly-deliverable optical fiber transducer 100. In an example, optical energy generated by the tunable optical source 204 can be coupled to a reference optical cavity 102, such as contemporaneously coupled to both the intravascularly-deliverable optical fiber transducer 100 and the reference optical cavity 102. A phase noise signature obtained from the reference optical cavity 102, less modulated or un-modulated by vibration, pressure, or strain coupled to the optical fiber transducer 100, can be subtracted from the optical energy reflected from the intravascularly-deliverable optical fiber transducer 100.

FIG. 6 illustrates generally an example of a system 600 that can include an intravascularly-deliverable optical fiber transducer 100, such as shown and described in the examples of FIGS. 1A, 1B, FIG. 2, or elsewhere herein. The system can include a tunable optical source 204, such as coupled to the intravascularly-deliverable optical fiber transducer 100, as discussed above in relation to the example of FIG. 5. The system 600 can include an optical path through an optical delay line 208 (e.g., an optical fiber configured to provide a specified optical propagation delay, or one or more other assemblies configured to delay an optical signal using a specified propagation delay), or a coupling 206 bypassing the optical delay line 208. The optical coupling 206 need not be entirely separate from an optical coupling including the optical delay line 208.

In an example, information can be obtained indicative of optical energy reflected from the intravascularly-deliverable optical fiber transducer 100 in response to an instance of optical energy generated by the tunable first optical source 204, bypassing the optical delay line 208, during a first duration. During a second duration, information can be obtained indicative of the optical energy reflected from the intravascularly-deliverable optical fiber transducer 100 in response to the same instance of optical energy generated by the tunable optical source 204, but such instance delayed by the delay line optical fiber for a duration defined by the specified optical propagation delay before being provided to the intravascularly-deliverable optical fiber transducer 100.

In an ultrasound imaging application, the specified optical propagation delay of the delay line 208 can correspond to at least a round-trip acoustic propagation delay between a specified ultrasonic imaging depth in an insonified tissue region nearby a portion of the intravascularly-deliverable optical fiber transducer 100 and the transducer 100.

During the first duration, a reflected portion of the optical energy generated by the tunable first optical source 204 and reflected from the intravascularly-deliverable optical fiber transducer 100 can include modulated optical energy corresponding to ultrasonic vibration of the intravascularly-deliverable optical fiber transducer 100 including ultrasonic energy reflected from the insonified region of tissue along with reflected optical energy indicative of a phase noise of the tunable first optical source 204.

During a second duration, such as in the absence of preceding acoustic energy transmission into the tissue, a phase noise signature can be obtained using information about reflected optical energy from the intravascularly-deliverable optical fiber transducer 100, less modulated or un-modulated by vibration, pressure, or strain coupled to the optical fiber transducer 100. As discussed elsewhere herein, information about such phase noise can be subtracted from information obtained during the first duration, indicative of the reflected from the intravascularly-deliverable optical fiber transducer 100.

For example, the information obtained during the first and second durations can be elicited in response to a signal instance of optical energy provided by the tunable first optical source 204, such as presented to the optical fiber transducer bypassing the delay line 208, and then presented again to the optical fiber transducer after propagating through the delay line 208. In this manner, an obtained time domain phase noise signature can be similar or identical during the first and second durations, because such durations correspond to the same or similar instances of optical energy output from the tunable first optical source 204.

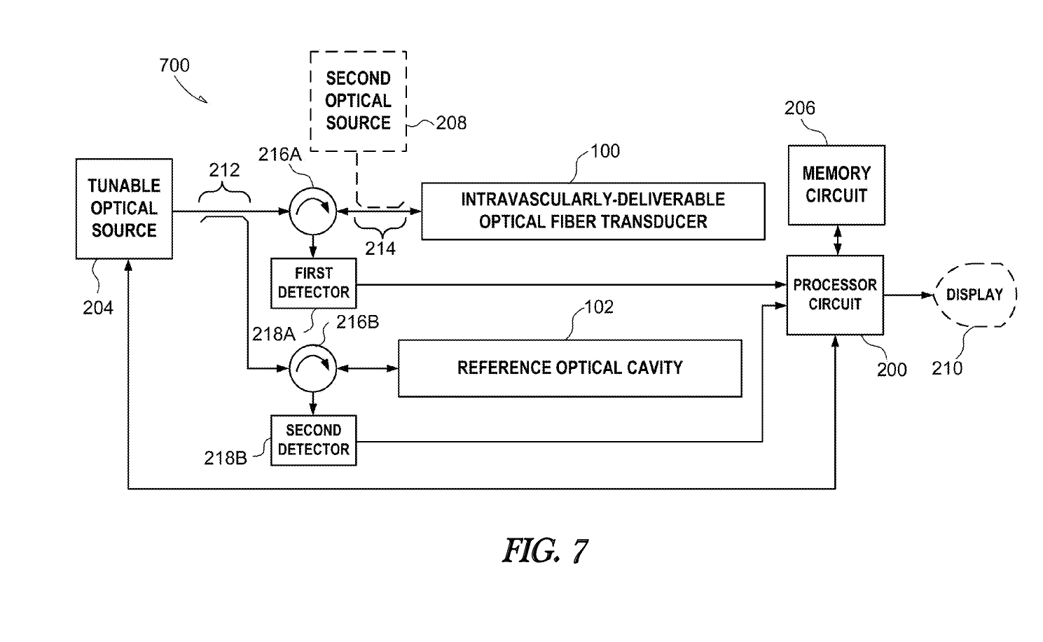

FIG. 7 illustrates generally an example of a system 700 that can include an intravascularly-deliverable optical fiber transducer 100 and a reference optical cavity 102. A tunable first optical source 204 can be coupled to the intravascularly-deliverable optical fiber transducer 100, such as through a first optical circulator 216A. Reflected optical energy modulated or adjusted by the intravascularly-deliverable optical fiber transducer 100 can be coupled back to a first detector 218A, such as through the first optical circulator 216A. Information indicative of the reflected optical energy can be provided to a processor circuit 200, such as for storage in a memory circuit 206.

The processor circuit 200 can be configured to process information from the reflected optical energy indicative of one or more of a vibration, pressure, or strain impressed upon or otherwise coupled to a sensing region of the intravascularly-deliverable optical fiber transducer 100, such as to determine one or more physiologic parameters or to construct an image of a physiologic region nearby the sensing region. Such physiologic parameters or imaging information can be presented to a user using a display 210.

In a mechanical vibration or acoustic (e.g., ultrasound) sensing application, a second optical source 208 can be included. The second optical source 208 can be configured for pulsed operation, such as including an optical output energy level higher than the level provided by the tunable optical source 204. The second optical source can be coupled to the intravascularly deliverable optical fiber transducer 100 such as using a first splitter/combiner 214. The second optical source 208 can be configured to generate a coherent optical output including optical energy having a different wavelength than a wavelength provided by the first optical source. For example, the second optical source can be configured to generate optical energy at a wavelength of about 1060 nanometers (nm) as compared to a wavelength of the tunable optical source 204. For example, the tunable optical source 204 can be configured to generate optical energy at a wavelength of around 1550 nm.

A region of tissue can be insonified, such as using energy obtained optically from the second optical source 208, such as discussed in relation to the example of FIG. 2 (e.g., using a blazed FBG to couple the optical energy to a photo-acoustic material). In response, acoustic energy reflected from the insonified region of tissue can be detected using the tunable optical source 204 to probe the intravascularly-deliverable optical fiber transducer 100, such as using the intravascularly-deliverable optical fiber transducer 100 to modulate or adjust the reflected optical energy, which can be detected by the first detector 218A and processed by the processor circuit 200. Acoustic energy used to insonify the region of tissue can include a center frequency of between about 5 MHz and about 50 MHz, or one or more other ranges of frequencies.

A second splitter/combiner 212 can coupled the optical energy from the tunable first optical source 204 to the reference optical cavity 102. As discussed in examples elsewhere herein, the reference optical cavity can include another optical fiber transducer similar or identical to the intravascularly-deliverable optical fiber transducer 100, or one or more other optical cavities, such as a temperature-controlled or temperature-stabilized ultra-high-finesse optical cavity (e.g., a cavity exhibiting a resonant response having a quality factor comparable to or exceeding a quality factor of an interferometer structure included as a portion of the intravascularly-deliverable optical fiber transducer 100).

An instance of optical energy can be coupled to the reference optical cavity 102 contemporaneously with (e.g., at the same time as) coupling to the intravascularly-deliverable optical fiber transducer 100. Optical energy reflected from the reference optical cavity 102 can be coupled to a second detector 216B, such as through the second circulator 218B. In this manner, a phase noise signature representative of the phase noise of the tunable optical source 204 can be provided to a processor circuit, such as to enhance information obtained from the intravascularly-deliverable optical fiber transducer 100. For example, a phase noise signature obtained from the reference optical cavity 102 can be one or more of normalized or aligned in time with information obtained from the optical fiber transducer 100, and the phase noise contribution can be subtracted or otherwise reduced or suppressed from the information obtained from the optical fiber transducer 100.

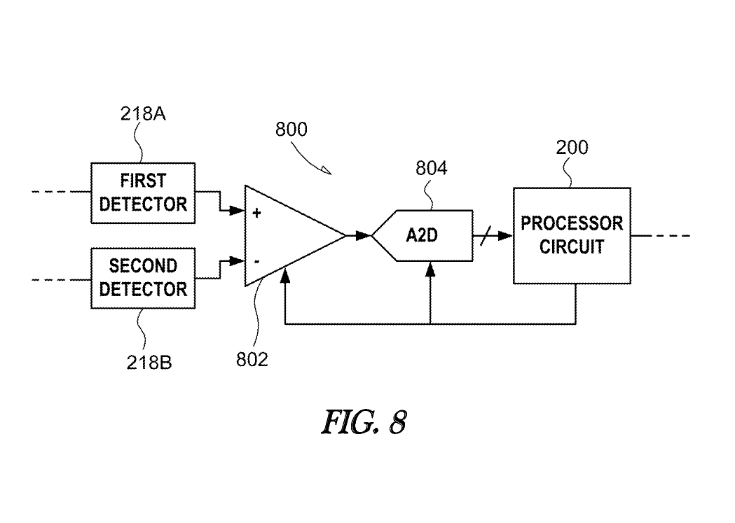

FIG. 8 illustrates generally an illustrative example of at least a portion of a system 800 that can include respective detectors coupled to an analog operational circuit 802. As discussed in examples elsewhere herein, information indicative of a phase noise of an optical source can be used to enhance information obtained from an intravascularly-deliverable optical fiber transducer. This enhancement can be performed using optical, analog electrical, or digital electrical techniques.

In an example, reflected optical energy including both a signal of interest (e.g., corresponding to acoustic or mechanical vibrational energy) and reflected energy indicative of the phase noise of the optical source can be detected using the first detector 218A. Reflected energy indicative of the phase noise of the optical source less modulated by signals of interest can be detected using the second detector 218B. In the example of FIG. 8, information obtained from a first optical detector 218A and information obtained from a second optical detector 218B can be fed into the operational circuit 802, such as to subtract the information obtained from the second detector 218B from the information obtained from the first detector 218A, and an output from the operation circuit can be digitized, such as using an analog-to-digital converter (A2D) 804. The digital output of the A2D 804 can be provided to a processor circuit, such as for extraction or determination of one or more physiologic parameters.

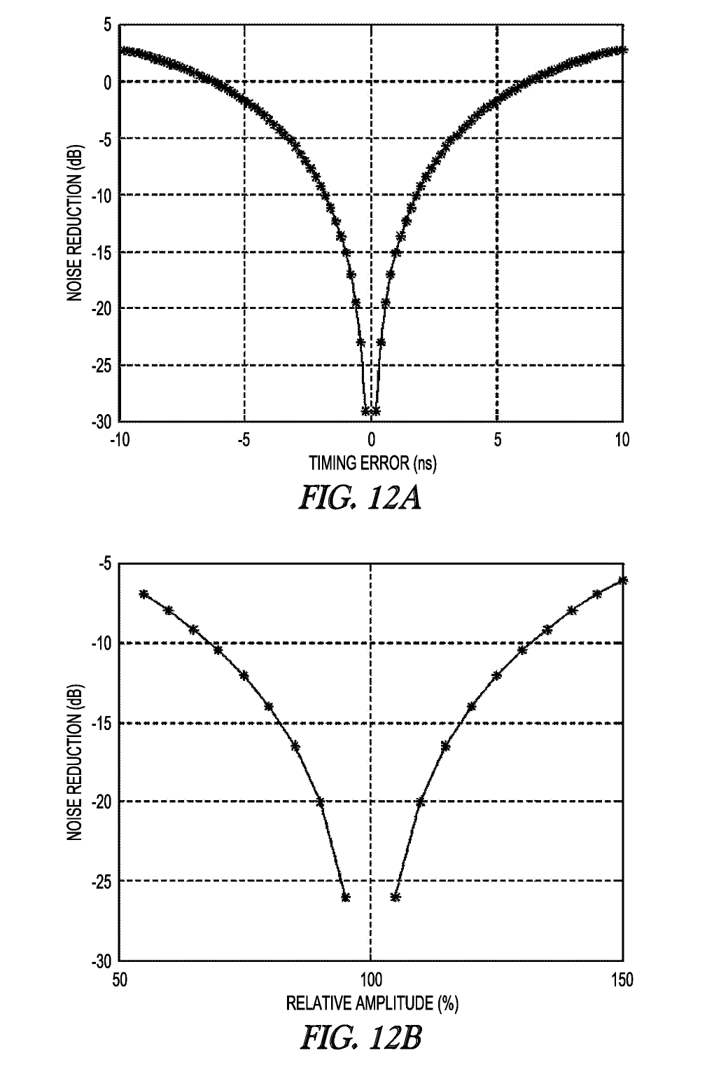

The example of FIG. 8 is illustrative. Other techniques can also be used to suppress or eliminate a time-domain phase noise signature from information of interest, such as including using one or more analog or digital electrical circuits, or an optical circuit, such as to determine a difference or other relative indication of information between respective optical signals provided to the first detector 218A or the second detector 218B, or to electrical signals obtained from the respective detectors. For example, an amplitude of detected information from the respective detectors 218A and 218B can be scaled or normalized, or aligned in time, as discussed in the examples of FIGS. 12A and 12B. Such scaling or alignment can be performed using analog or digital techniques.

In addition, or alternatively, delay compensation or timing alignment can be performed in the optical domain, such as using one or more optical delay lines, such as to equalize or align optical path delays in each “arm” of the system. For example, a first “arm” can include the optical fiber transducer, and a second “arm” can include the optical reference cavity, such as corresponding to the examples of FIGS. 5 and 7. FIG. 12B provides an illustrative example of a comparison between noise reduction performance as a function of a timing alignment error between a demodulated phase noise signature and a received signal from the optical fiber transducer that can include both a signal of interest and demodulated phase noise. For example, the phase noise signature obtained from the reference cavity can be aligned or scaled and then subtracted in the time domain from the reflected optical energy from the optical fiber transducer to reduce or suppress (e.g., cancel) the phase noise contribution to the energy obtained from the optical fiber transducer, such as improving a system SNR.

In an illustrative example, one or more optical delay lines can be controllably inserted in an optical path of the optical fiber transducer or reference cavity arms, such as to provide a selectable optical delay in increments of about 1/16th of a wavelength of an envelope of an acoustic signal modulating the reflected optical energy from the optical fiber transducer. In an illustrative example including an ultrasound application, the acoustic energy can have a center frequency of about 25 MHz, and the corresponding optical delay line increment can include an optical path length increment of about 20 centimeters (cm). For example, delay lines including integer multiples of such a path length increment can be controllably added or removed to an optical path in a specified “arm” of the system to equalize a an optical path length or otherwise temporally align phase noise information reflected in the respective arms to respective detectors.

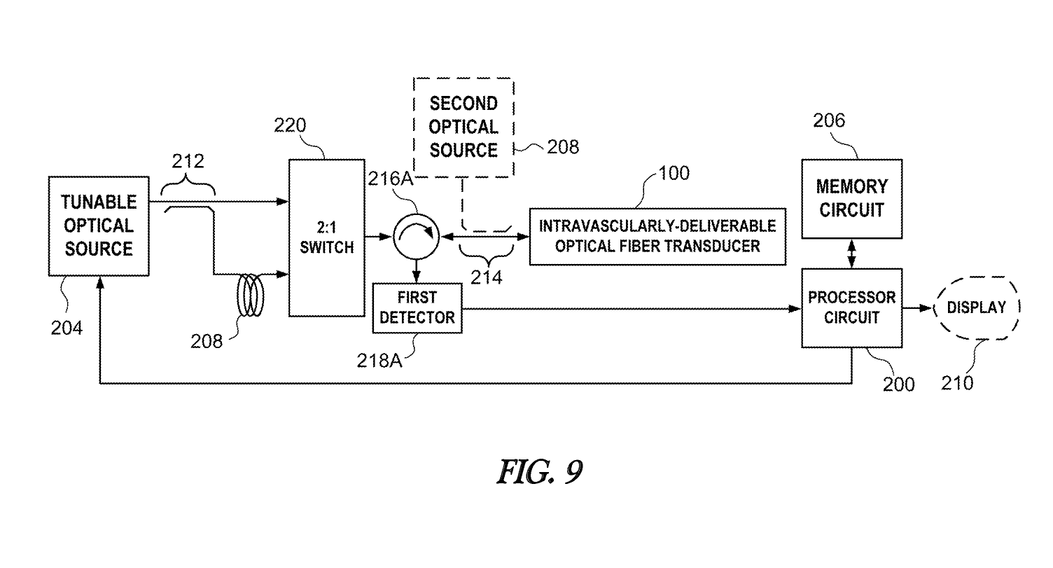

FIG. 9 illustrates generally an example of a system that can include intravascularly-deliverable optical fiber transducer 100 and an optical delay line 208. Similar to the example of FIG. 6, or portions of examples described elsewhere herein, the system 600 can include a tunable first optical source 204, such as coupled to a two-to-one (2:1) optical switch 220. The tunable first optical source 204 can be coupled to a first input of the switch 220 bypassing an optical delay line 208, or coupled through the delay line 208 to a second input of the switch 220, such as using a splitter/combiner 212. An output of the switch 220 can be coupled to an optical circulator 216A.

Optical energy reflected from the intravascularly-deliverable optical fiber transducer 100 can be coupled to a first detector 218A. As discussed in other examples herein, a second optical source 208 can provide optical energy to the optical fiber transducer 100 such as using a splitter/combiner 214 (e.g., output from the tunable optical source 204 and the second optical source 208 can be wavelength-division-multiplexed (WDM)). Such optical energy from the second optical source 208 can be converted to an acoustic transmit pulse to elicit reflection of acoustic energy from structures in a tissue region nearby an acoustically-emitting portion of the optical fiber transducer 100. Such reflections can be converted from optical to electrical signals using the first detector 218A, such as for processing by a processor circuit 200, or storage in a memory circuit 206. An image or other information indicative of the optical energy reflected from the optical transducer 100 can be presented to a user, such as using a display 210.

As discussed in other examples elsewhere herein, an instance of optical energy from the tunable first optical source 204 can be coupled to both the first instance of the optical switch 220, and to the delay line 208. In this manner, a time-domain phase noise signature from the tunable first optical source 204 can be obtained during respective first and second durations. In an example, such as during the first duration, the instance of optical energy from the tunable first optical source 204 can be presented to the optical fiber transducer 100 during or shortly after a “transmit pulse” from the second optical source 208. During a second duration, the instance of optical energy from the tunable first optical source 204 can be delayed for a specified duration by the optical delay line 208, and then presented to the optical fiber transducer 100, such as without a corresponding transmit pulse. In this manner, during the second duration, the reflected optical energy can be less modulated or un-modulated by vibration, stress, or strain, and can provide information indicative primarily of the phase noise of the tunable optical source 204. The first and second durations need not occur in any particular order. For example, a transmit pulse can be suppressed during the first duration, the phase noise signature can be obtained, and the transmit pulse can be enabled shortly before or during the second duration.

In an illustrative example, the specified optical propagation delay of the optical delay line 208 can be about 6 microseconds (μs), such as corresponding to an optical fiber length of about 1.2 kilometers (km). A latency of the 2:1 switch 220 can be about 1 μs or less.

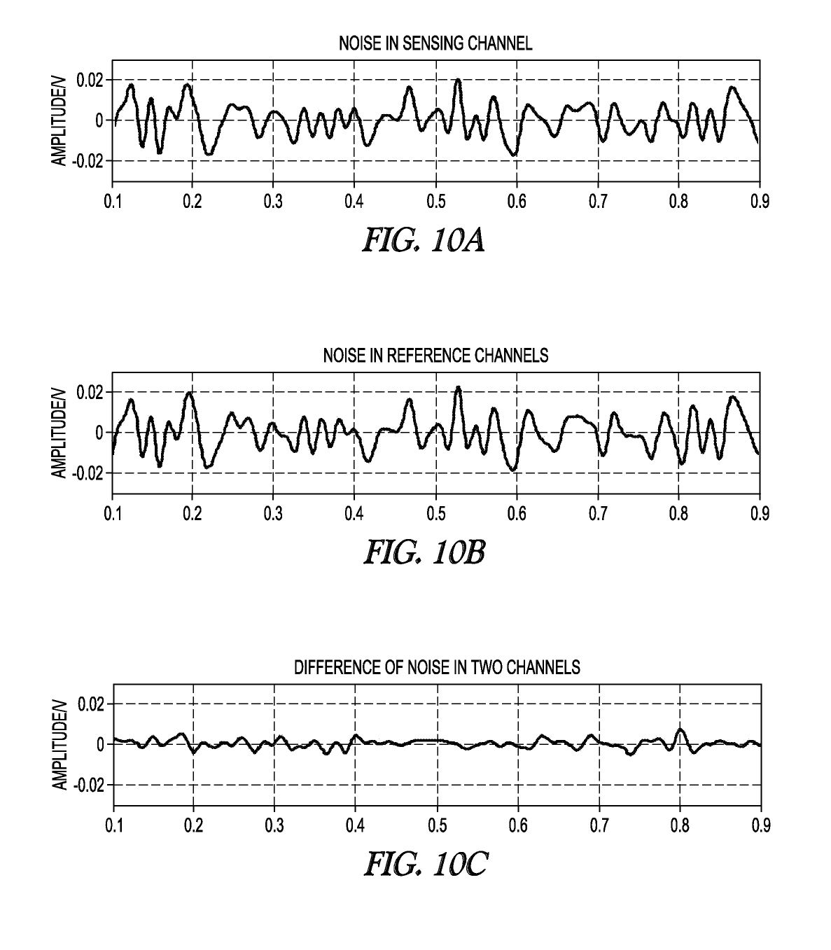

FIGS. 10A through 10C illustrate generally illustrative examples of a noise signature that can be obtained from a sensing channel (e.g., FIG. 10A, such as corresponding to a first “arm” of the examples of FIGS. 5 and 7), a noise signature that can be obtained from a reference channel (e.g., FIG. 10B, such as corresponding to a second “arm” of the examples of FIGS. 5 and 7, including the reference cavity), and a relative indication of information that can be obtained such as using a difference between the signatures obtained from the sensing and reference channels (FIG. 10C).

In the illustrative examples of FIGS. 10A through 10C, both FIGS. 10A and 10B include information indicative of a phase noise signature less modulated or un-modulated by strain, pressure, or vibration coupled to the optical fiber transducer (in FIG. 10A), or the reference cavity (in FIG. 10B). FIG. 10C shows that the phase noise can be substantially suppressed, such as after aligning and scaling one or more of the signatures in FIGS. 10A and 10B and then obtaining a difference (e.g., a subtraction in the time domain, such as using an analog electrical circuit, or subtraction of corresponding samples in a time series in the digital domain).

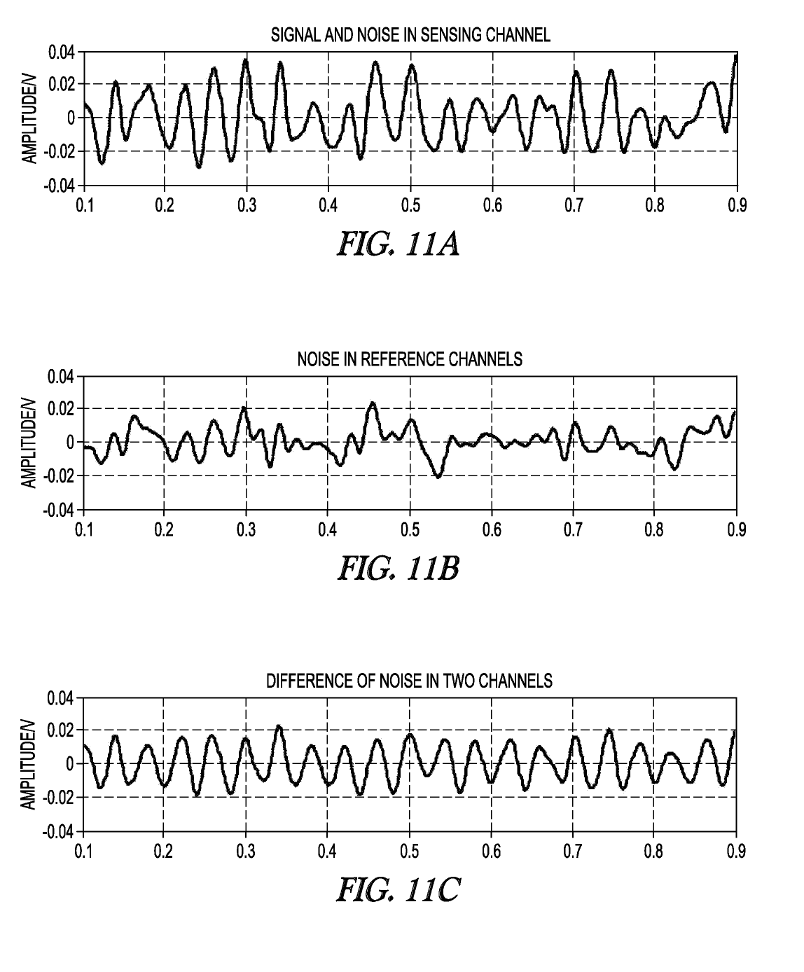

FIGS. 11A through 11C illustrate generally illustrative examples of a signal of interest plus a noise signature (in FIG. 11A) that can be obtained from a sensing channel (e.g., a first “arm” of the examples of FIGS. 5 and 7), a noise signature (in FIG. 11B) that can be obtained from a reference channel (e.g., a second “arm” of the examples of FIGS. 5 and 7), and a relative indication of information that can be obtained such as using a difference between the signatures obtained from the sensing and reference channels (in FIG. 11C).

Such techniques are generally applicable to the delay-line based techniques and apparatus, such as discussed in relation to the examples of FIGS. 6 and 9, where a phase noise profile can be obtained such as using apparatus including an optical delay line.

In the illustrative example of FIGS. 11A through 11C, an optical fiber transducer can be immersed in a water tank. A 25 MHz acoustic signal can be coupled to the water tank. Information can be obtained indicative of the optical energy reflected from the optical fiber transducer, including both a signal of interest (e.g., the 25 MHz sinusoidal vibration signal) along with a phase noise signature superimposed on the vibration signal. Information representative of the phase noise signature can be obtained, such as shown in FIG. 11B. The phase noise signature information can then be subtracted from the information in FIG. 11A, such as to recover information corresponding to 25 MHz sinusoidal vibration signal, with the phase noise reduced or suppressed, as shown in FIG. 11C.

In the illustrative examples of FIGS. 11A through 11C, the reference cavity can include a second optical fiber transducer similar to the optical fiber transducer immersed in the water tank. However, other reference cavities can be used, such as temperature-stabilized or temperature-controlled tunable optical cavity, such as an ultra-high-finesse optical cavity.

FIG. 12A illustrates generally an illustrative example of noise reduction performance in decibels as a function of a timing alignment error between phase noise information in a signal of interest as compared to information about phase noise that can be obtained separately using a reference cavity or delay line. In an example including a time-domain subtraction, if information indicative of phase noise (e.g., a phase noise signature, such as shown in FIG. 10B or 11B) is misaligned in time with corresponding phase noise imposed on the signal of interest, noise reduction performance can be degraded.

FIG. 12B illustrates generally an illustrative example of noise reduction performance as a function of a amplitude normalization error between phase noise information in a signal of interest as compared to a phase noise obtained using a reference cavity or delay line. Similar to the illustrative example of FIG. 12B, if an amplitude of a phase noise signature (e.g., as shown in the examples of FIG. 10B or 11B) is not the same as a corresponding phase noise contribution to a signal of interest, noise reduction performance can be degraded. Either or both the signal of interest or the phase noise signature can be adjusted (e.g., amplified or attenuated in the analog or optical domain, or scaled in the digital domain), such as to substantially match the amplitude of the phase noise signature with corresponding phase noise in a signal of interest.

For example, such a scaling factor can be adjusted or determined such as obtaining a “baseline” from a first arm substantially un-modulated by vibration, strain, or pressure (or using information obtained during a first duration). A phase noise signature can be obtained from a second arm (or using information obtained during a second duration), and the information obtained can be appropriately scaled to substantially equalize the resulting amplitudes. Such a scaling factor can then be used for subsequent sensing.

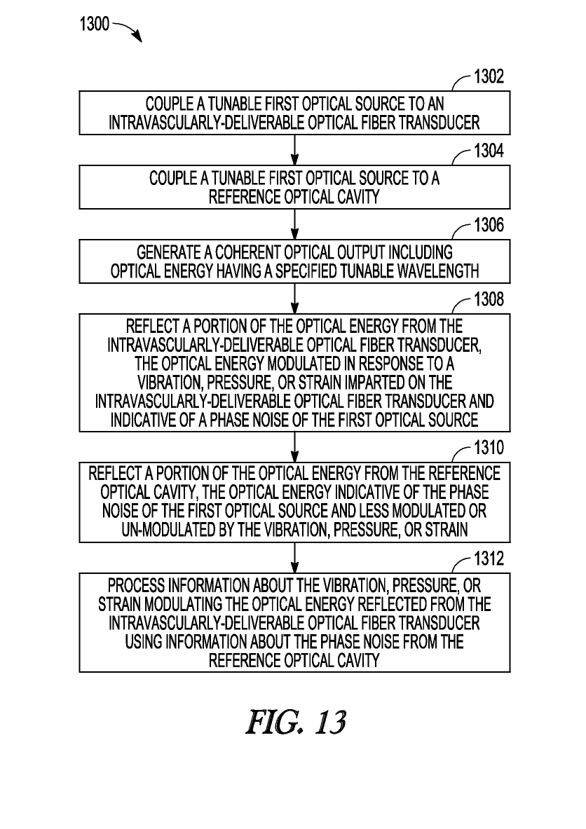

FIG. 13 illustrates generally a technique 1300, such as a method, that can include processing information about a vibration, pressure, or strain modulating optical energy reflected from an intravascularly-deliverable optical fiber transducer using information about a phase noise of an optical source obtained from a reference optical cavity. The technique of FIG. 13 can be used, such as with the apparatus discussed elsewhere herein, for example, in the examples of FIGS. 5 and 7.

At 1302, a tunable first optical source can be coupled to an intravascularly-deliverable optical fiber transducer. At 1304, the tunable first optical source can be coupled to a reference optical cavity. At 1306, a coherent optical output can be generated, such as using the tunable first optical source, including optical energy having a specified tunable wavelength.

At 1308, a portion of the optical energy can be reflected from the optical fiber transducer, such as modulated in response to a vibration, pressure, or strain imparted on the optical fiber transducer, and including reflected optical energy indicative of a phase noise of the tunable first optical source.

At 1310, a portion of the optical energy can be reflected from the reference optical cavity, the optical energy indicative of the phase noise of the tunable first optical source and less modulated or un-modulated by the vibration, pressure, or strain.

At 1312, information about the vibration, pressure, or strain modulating the optical energy can be processed using the information about the phase noise from the reference optical cavity. For example, the phase noise information can be subtracted from the information obtained from the optical fiber transducer to improve an SNR of a signal of interest obtained from the optical energy reflected from the optical fiber transducer.

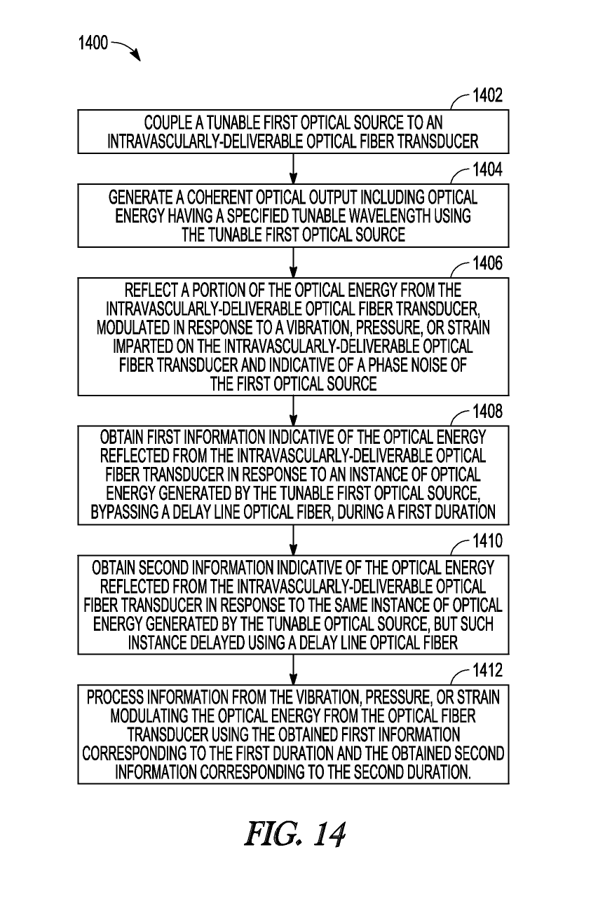

FIG. 14 illustrates generally a technique 1400, such as a method, that can include processing information about a vibration, pressure, or strain modulating optical energy reflected from an intravascularly-deliverable optical fiber transducer using information about a phase noise of an optical source obtained as least in part using an optical delay line.

At 1402, a tunable first optical source can be coupled to an intravascularly-deliverable optical fiber transducer. At 1404, a coherent optical output can be generated, such as using the tunable first optical source, including optical energy having a specified tunable wavelength.

At 1406, a portion of the optical energy can be reflected from the optical fiber transducer, such as modulated in response to a vibration, pressure, or strain imparted on the optical fiber transducer, and including reflected optical energy indicative of a phase noise of the tunable first optical source.

At 1408, first information indicative of the optical energy reflected from intravascularly-deliverable optical fiber transducer can be obtained, such as in response to an instance of optical energy generated by the tunable first optical source. For example, such information can correspond to delivery of the instance of optical energy through an optical pathway bypassing an optical delay line, during the first duration.

At 1410, second information indicative of the optical energy reflected from intravascularly-deliverable optical fiber transducer can be obtained, such as in response to the same instance of optical energy generated by the tunable optical source, but delayed using the optical delay line.

At 1412, information indicative of a vibration, pressure, or strain modulating the optical energy reflected from the optical fiber transducer can be processed, using the obtained information corresponding to the first and second durations. For example, a phase noise signature can be obtained, corresponding to information obtained during the first or second duration, and such a phase noise signature can be subtracted from other information, such as information indicative of vibration, pressure, or strain, such as to reduce or suppress phase-noise-induced degradation of the information indicative of vibration, pressure, or strain.

### Various Notes & Examples

Each of the non-limiting examples included in this document can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. An optical system, comprising:
an intravascularly-deliverable optical fiber transducer, configured to be coupled to a tunable first optical source configured to generate a coherent optical output including optical energy having a specified tunable wavelength, the intravascularly-deliverable optical fiber transducer configured to reflect a portion of the optical energy modulated in response to a vibration, pressure, or strain;
a delay line optical fiber including a length defining a specified optical propagation delay, the delay line optical fiber configured to be coupled the tunable first optical source and configured to be coupled to the intravascularly-deliverable optical fiber transducer;
a processor circuit configured to:
obtain first information indicative of the optical energy reflected from the intravascularly-deliverable optical fiber transducer in response to an instance of optical energy generated by the tunable first optical source, bypassing the delay line optical fiber, during a first duration;
obtain second information indicative of the optical energy reflected from the intravascularly-deliverable optical fiber transducer in response to the same instance of optical energy generated by the tunable optical source, but such instance delayed by the delay line optical fiber for a duration defined by the specified optical propagation delay before being provided to the intravascularly-deliverable optical fiber transducer during a second duration, the specified optical propagation delay corresponding to at least a round-trip acoustic propagation delay between a specified ultrasonic imaging depth in an insonified tissue region and a portion of intravascularly-deliverable optical fiber transducer configured to insonify the region of tissue; and
process information from the vibration, pressure, or strain modulating the optical energy reflected from the optical fiber transducer using the obtained first information corresponding to the first duration and the obtained second information corresponding to the second duration.

2. The optical system of claim 1, wherein the intravascularly-deliverable optical fiber transducer is coupleable to a second optical source configured to generate a coherent optical output including optical energy having a different wavelength than the wavelength provided by the tunable first optical source, the intravascularly-deliverable optical fiber transducer configured to insonify a region of tissue ultrasonically using energy obtained optically from the second optical source;
wherein the reflected portion of the optical energy generated by the tunable first optical source and reflected from the intravascularly-deliverable optical fiber transducer obtained during the first duration includes modulated optical energy corresponding to ultrasonic vibration of the intravascularly-deliverable optical fiber transducer including ultrasonic energy reflected from the insonified region of tissue and optical energy indicative of the phase noise of the tunable first optical source; and
wherein the reflected portion of the optical energy from the intravascularly-deliverable optical fiber transducer obtained during the second duration includes optical energy indicative of the phase noise of the tunable first optical source and less modulated or un-modulated by the vibration, pressure, or strain.

3. The optical system of claim 2, further comprising the second optical source configured to generate the coherent optical output including the optical energy having a different wavelength than the wavelength provided by the tunable first optical source

4. The optical system of claim 2, wherein the processor circuit is configured to process information from the vibration, pressure, or strain modulating the optical energy from the intravascularly-deliverable optical fiber transducer by subtracting a representation of the second information from a representation of the first information.

5. The optical system of claim 4, wherein the processor circuit is configured to process information from the vibration, pressure, or strain modulating the optical energy from the intravascularly-deliverable optical fiber transducer by aligning the respective representations of the first and second information in time prior to the subtracting.

6. The optical system of claim 4, wherein the processor circuit is configured to process information from the vibration, pressure, or strain modulating the optical energy from the intravascularly-deliverable optical fiber transducer by scaling one or more of the respective amplitudes of the respective representations of the first and second information prior to the subtracting.

7. The optical system of claim 1, further comprising the tunable first optical source configured to generate the coherent optical output including optical energy having the specified tunable wavelength.

8. The optical system of claim 7, wherein the intravascularly-deliverable optical fiber transducer includes respective first and second optical fiber Bragg grating (FBG) structures, the first FBG structure located more proximally to the first optical source than the second FBG structure along the length of the intravascularly-deliverable optical fiber transducer, and the first and second FBG structures configured to define a first interferometer structure.

9. The optical system of claim 8, wherein the specified tunable wavelength provided by the first optical source is established by locking the first optical source to a transmission feature of the first interferometer structure.

10. The optical system of claim 9, wherein the specified tunable wavelength provided by the tunable first optical source is established by the locking the tunable first optical source to a wavelength about halfway along a transition between a relative minimum intensity of optical energy reflected from the interferometer structure and a wavelength corresponding to a relative maximum intensity of optical energy reflected from the first interferometer structure.

11. The optical system of claim 1, comprising:
an optical detector coupled to the intravascularly-deliverable optical fiber transducer and configured to provide the information to the processor circuit, the information indicative of the optical energy reflected from the intravascularly-deliverable optical fiber transducer; and
an optical switch configured to provide (1) a first optical path from the tunable first optical source to the intravascularly-deliverable optical fiber transducer including the delay line optical fiber during the first duration and (2) a second optical path bypassing the delay line optical fiber during the second duration.

12. An method, comprising:
coupling a tunable first optical source to an intravascularly-deliverable optical fiber transducer;
generating a coherent optical output including optical energy having a specified tunable wavelength using the tunable first optical source;
reflecting a portion of the optical energy from the intravascularly-deliverable optical fiber transducer, the optical energy modulated in response to a vibration, pressure, or strain imparted on the intravascularly-deliverable optical fiber transducer and reflecting a portion of the optical energy indicative of a phase noise of the first optical source;
obtaining first information indicative of the optical energy reflected from the intravascularly-deliverable optical fiber transducer in response to an instance of optical energy generated by the tunable first optical source, bypassing a delay line optical fiber, during a first duration;
obtaining second information indicative of the optical energy reflected from the intravascularly-deliverable optical fiber transducer in response to the same instance of optical energy generated by the tunable optical source, but such instance delayed using a delay line optical fiber including a length establishing a specified optical propagation delay, during a second duration, the specified optical propagation delay corresponding to at least a round-trip acoustic propagation delay between a specified ultrasonic imaging depth in an insonified tissue region and a portion of intravascularly-deliverable optical fiber transducer configured to insonify the region of tissue; and
using a processor circuit, processing information from the vibration, pressure, or strain modulating the optical energy from the optical fiber transducer using the obtained first information corresponding to the first duration and the obtained second information corresponding to the second duration.

13. The method of claim 12, comprising:
coupling the intravascularly-deliverable optical fiber transducer to a second optical source;
using the second optical source, generating a coherent optical output including energy having a different wavelength than the wavelength provided by the tunable first optical source; and
in response, using the intravascularly-deliverable optical fiber, insonifying a region of tissue ultrasonically using energy obtained optically from the second optical source;
wherein the reflecting a portion of the optical energy from the intravascularly-deliverable optical fiber transducer includes:
during the first duration, reflecting modulated optical energy corresponding to ultrasonic vibration of the intravascularly-deliverable optical fiber transducer including ultrasonic energy reflected from the insonified region of tissue and optical energy indicative of the phase noise of the tunable first optical source; and
during the second duration, reflecting optical energy indicative of the phase noise of the tunable first optical source and less modulated or un-modulated by the vibration, pressure, or strain.

14. The method of claim 12, wherein the processing information from the vibration, pressure, or strain modulating the optical energy from the intravascularly-deliverable optical fiber transducer includes subtracting a representation of the second information from a representation of the first information.

15. The method of claim 14, wherein the processing information from the vibration, pressure, or strain modulating the optical energy from the intravascularly-deliverable optical fiber transducer includes aligning the respective representations of the first and second information in time prior to the subtracting.

16. The method of claim 14, wherein the processing information from the vibration, pressure, or strain modulating the optical energy from the intravascularly-deliverable optical fiber transducer includes scaling one or more of the respective amplitudes of the respective representations of the first and second information prior to the subtracting.

17. The method of claim 12, wherein the intravascularly-deliverable optical fiber transducer includes respective first and second optical fiber Bragg grating (FBG) structures, the first FBG structure located more proximally to the first optical source than the second FBG structure along the length of the intravascularly-deliverable optical fiber transducer, and the first and second FBG structures configured to define a first interferometer structure.

18. The method of claim 17, comprising establishing the specified tunable wavelength provided by the first optical source including locking the first optical source to a transmission feature of the first interferometer structure.

19. The method of claim 18, wherein establishing the specified tunable wavelength comprises locking the tunable first optical source to a wavelength about halfway along a transition between a relative minimum intensity of optical energy reflected from the interferometer structure and a wavelength corresponding to a relative maximum intensity of optical energy reflected from the first interferometer structure.

20. The method of claim 12, comprising:
using an optical switch, providing (1) a first optical path from the tunable first optical source to the intravascularly-deliverable optical fiber transducer including the delay line optical fiber during the first duration and (2) a second optical path bypassing the delay line optical fiber during the second duration;
obtaining information indicative of the optical energy reflected from the intravascularly-deliverable optical fiber transducer using a first optical detector; and
providing the information to the processor circuit, the information indicative of the optical energy reflected from the intravascularly-deliverable optical fiber transducer.
